(19) United States
(12) Plant Patent Application Publication
(10) Pub. No.: US 2003/0046737 P1
(43) Pub. Date: Mar. 6, 2003

(54) Shrub rose plant named Ausecret

(75) Inventor: David C.H. Austin, Albrighton (GB)

Correspondence Address: KLARQUIST SPARKMAN CAMPBELL LEIGH & WHINSTON, LLP, One World Trade Center, Suite 1600, 121 S.W. Salmon Street, Portland, OR, 97204, US

(73) Assignee: David Austin Roses

(21) Appl. No.: 09/947,252

(22) Filed: Sep. 5, 2001

(51) Int. Cl. A01H 5/00

(52) U.S. Cl. PLT/107

(57) ABSTRACT

Shrub rose plant having short spreading growth; warm pink rosette-shaped flowers produced in quite large sprays; light fruity type fragrance; and soft green, highly polished foliage.

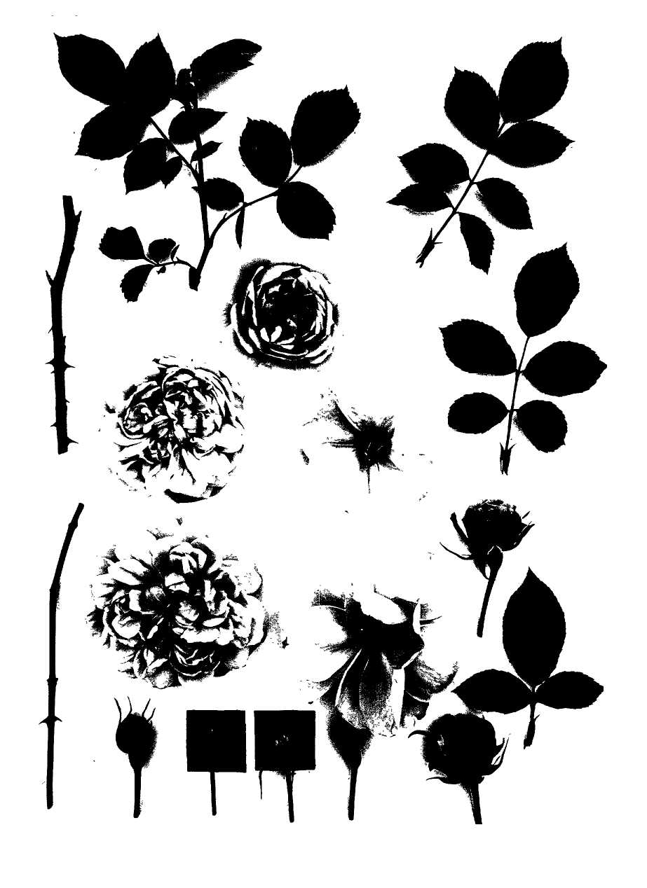

### SUMMARY OF THE INVENTION

The present invention relates to a new and distinct variety of rose plant of the shrub class which was originated by me by crossing the variety AUSmas (unpatented), with an unnamed seedling (unpatented).

### BACKGROUND OF THE INVENTION

The primary objective of this breeding was to produce a new disease resistant, free flowering rose variety with flowers of the classic English rose shape and with a good fragrance.

### BRIEF SUMMARY OF THE INVENTION

The objective was substantially achieved, along with other desirable improvements, as evidenced by the following unique combination of characteristics that are outstanding in the new variety and that distinguish it from its parents, as well as from all other varieties of which I am aware:

1. Short spreading growth;

2. Warm pink rosette-shaped flowers produced in quite large sprays;

3. Light fruity type fragrance; and

4. Soft green, highly polished foliage.

Asexual reproduction of this new variety by budding, as performed at Bowling Green Lane, shows that the foregoing and all other characteristics and distinctions come true to form and are established and transmitted through succeeding propagations.

### COMPARISON WITH PARENTS

The seed parent AUSmas has a full, upright growth habit and bears rich yellow flowers. The pollen parent has a more upright and bushy growth habit and bears bright pink blooms. AUSecret has a medium arching growth habit and bears larger, soft pink flowers having more petals than those of the pollen parent.

### COMPARISON WITH SIMILAR VARIETY

The closest variety is AUSmak which has a more upright bushy and branching growth habit, with leaves that are less shiny than AUSecret.

### BRIEF DESCRIPTION OF THE ILLUSTRATION

The accompanying illustration shows typical specimens of the vegetative growth and flowers of this new variety in different stages of development, depicted in color as nearly true as it is reasonably possible to make the same in a color illustration of this character.

### DETAILED BOTANICAL DESCRIPTION

The following is a detailed description of my new rose cultivar with color descriptions using terminology in accordance with the Royal Horticultural Society (London) colour chart, except where ordinary dictionary significance of color is indicated.

Parentage:

Seed Parent.—AUSmas (unpatented).

Pollen Parent.—Unnamed seedling (unpatented).

Classification:

Botanical.—Rosa hybrida.

Commercial.—Shrub.

### FLOWER

Observations made in July, 1997, from plants of six years of age budded on Rosa dumetorium laxa rootstock, grown in a garden environment at David Austin Roses Ltd. in Bowling Green Lane, Albrighton, England. In the United States Mr. Huey rootstock is recommended.

Blooming Habit: Recurrent.

Bud:

Size.—.5-.75 inches long when the petals start to unfurl.

Form.—The bud form is average, pointed ovoid.

Color.—When sepals first divide, bud color is Red Group 38A. When half blown, the upper sides of the petals are Red-Purple group 41C; and the lower sides of the petals are Red-Purple Group 38A to Red Group 41D.

Sepals.—Color: Yellow-Green Group 144AB with RedPurple Group 60A blush. Length: 1 inch. Width: Between 0.4-0.5 inches. Shape: Spherical. Surface texture: Slightly hairy edges; mostly smooth with a waxy underneath. There are three lightly appendaged sepals. There are two unappendaged sepals with hairy edges.

Receptacle.—Color: Yellow-Green Group 144AB with Red-Purple Group 60A blush. Shape: Pitcher. Size: Medium, broad, long, about 0.3-0.6 inches×0.3-0.5 inches. Surface: Smooth.

Peduncle: Length: Medium, varying from 1 to 2.5 inches. Surface: Rough, glandular. Color: Yellow-Green Group 144B with GreyedPurple Group 184B overcoat. Strength: Stiff.

Bloom:

Size.—Medium. Average open diameter is about 3 inches.

Borne.—Several together in rounded clusters of 4 to 14 blooms.

Stems.—Strength Strong. Average length is about 20-24 inches.

Form.—When first open: Cupped. Permanence: Flattens, outer petals curl back.

Petalage.—Number of petals under normal conditions: 110.

Color.—The upper sides of the petals are Red-Purple Group 65C on outer portion; Red-Purple Group 65D in middle portion; and Yellow Group 8D at inner portion, the tip being Yellow Group 8A or 8B. The reverse sides of the petals are Red-Purple Group 65B on outer portion; Red-Purple Group 65B in middle portion; and Yellow Group 8D in lower portion with a Yellow Group 8B tip. The major color on the upper side is Red Group 51D on inner petals; and Red-Purple Group 65D on outer petals.

Variegations.—N/A.

Discoloration.—The general tonality at the end of the first day: RedPurple Group 65D. At the end of the third day: Red Group 56D.

Fragrance.—Moderate, fruity.

Petals:

Texture.—Thick.

Shape.—Outer petals are round and wide pointed. Inner petals are oval with a narrow point.

Margin.—Absent or very weak reflexing; weak undulation.

Apex Shape.—Heart shaped.

Base Shape.—Emarginate.

Form.—Tips slightly recurved.

Arrangement.—Quartered. Many petaloids are Red Group 41B in the center.

Persistence.—Petals drop off cleanly before drying.

Lastingness.—On the plant: Fair (about 3-4 days). As a cut flower: Short (about 2 days).

Reproductive Parts:

Anthers.—Size: Length: Medium (0.08 inch). Color: Yellow Group 6C. Arrangement: Mixed with petaloids.

Filaments.—Color: Yellow Group 8C.

Pollen.—Color: Yellow-Orange Group 15B.

Stamens.—Number: Few (30).

Pistils.—Number: 7. Styles: Color: Yellow-Green Group 154C Length: Short (about 0.1 inch) Stigmas: Color: Yellow Group 7C Length: 0.04 inch.

Hips.—Color: Yellow Group 14B. Shape: Small cup. Size: 0.3 inch -0.6 inch.

### PLANT

Form.—Shrub, English.

Growth.—Medium, sprawling.

Mature Plant.—At maturity, height is 3 feet, width is 3 feet.

Foliage: Number of leaflets on normal mid-stem leaves: 7.

Size.—Medium (About 2-2.75 inches long×1-1.5 inches wide).

Quantity.—Normal.

Color.—New foliage: Upper side: Yellow-Green Group 146A Lower side: Yellow-Green Group 146B. Old foliage: Upper side: Yellow-Green Group 147A. Lower side: Yellow-Greeen Group 147B.

Leaflets:

Size.—About 2 inches long×1 inch wide.

Shape.—Broadly oval.

Tip Shape.—Aristate (into a point).

Apex Shape.—Obtuse.

Texture.—Upper side: Leathery.

Edge.13 Serrated.

Serrations.—Small.

Petiole Color.—Yellow-Green Group 146C.

Petiole Rachis.—Color: Yellow-Green Group 144B. Underside: Smooth with prickles.

Auricle.—½inch long to the point.

Stipules.—Serrated. Length: About 1 inch.

Disease Resistance.—Resistant to mildew, blackspot and rust under normal growing conditions.

Pest Resistance.—Strong.

Wood:

New Wood.—Color: Yellow-Green Group 146A with GreyedPurple Group 184B blush. Bark: Smooth.

Old Wood.—Color: Green Group 143B. Bark: Smooth.

Prickles (Thorns):

Quantity.—On main canes from base: Ordinary. On laterals from main canes: Ordinary.

Form.—Hooked downward. Length: 0.2 inch.

Color when Young.—Greyed-Purple Group 184B.

Color when Mature.—Greyed-Purple Group 185A. Small Prickles:

Quantity.—On main stalks: None. On laterals: Few.

Color.—Red Group 47B.

Winter Hardiness: Hardy to zone 4 to 5.

I claim:

1. A new and distinct variety of rose plant of the shrub class, substantially as herein shown and described, characterized particularly by its short spreading growth; warm pink rosette-shaped flowers produced in quite large sprays; light fruity type fragrance; and soft green, highly polished foliage.
